(19) United States
(12) Patent Application Publication
(10) Pub. No.: US 2022/0168132 A1
(43) Pub. Date: Jun. 2, 2022

(54) OSTOMY EXPANSION PANEL

(71) Applicant: Custom Ostomy Clothing LLC, Detroit, MI (US)

(72) Inventor: Jo Anne JEWELL, Detroit, MI (US)

(21) Appl. No.: 17/540,420

(22) Filed: Dec. 2, 2021

Related U.S. Application Data
(60) Provisional application No. 63/120,295, filed on Dec. 2, 2020.

(51) Int. Cl. A61F 5/449; A41D 13/12

(52) U.S. Cl. CPC A61F 5/449; A41D 13/1254

(57) ABSTRACT

A garment includes an ostomy support garment that includes one or more slits configured to be adjacent to the front of an ostomy pack of a user, wherein the one or more slits are configured to open upon the ostomy pack being filled. The garment further includes an elastic material portion located on a back side of the garment and at least covering the one or more slits, wherein the elastic material portion is located adjacent to the front of the ostomy pack.

### CROSS-REFERENCE TO RELATED APPLICATIONS

This application claims the benefit and priority of U.S. Provisional Application 63/120,295 filed Dec. 2, 2020, the entire disclosure of which is incorporated by reference herein.

### TECHNICAL FIELD

The present disclosure relates to clothing and apparel, including those for people that have an ostomy.

### BACKGROUND

People wear garments every day for numerous activities, leisure wear, sports, business, casual, etc. However, many people require an ostomy. Most garments are not made with the segment of population in mind that require an ostomy. Thus, most mainstream garments may create comfort issues for people that require an ostomy.

### SUMMARY

A first embodiment illustrates a garment includes an ostomy support garment that includes one or more slits configured to be adjacent to the front of an ostomy pack of a user, wherein the one or more slits are configured to open upon the ostomy pack being filled. The garment further includes an elastic material portion located on a back side of the garment and at least covering the one or more slits, wherein the elastic material portion is located adjacent to the front of the ostomy pack.

A second embodiment, illustrates a garment that includes an ostomy support garment in the form of a material having an ostomy extension portion (“OEP”) that includes one or more slits configured to be adjacent to an ostomy pack of a user, wherein the one or more slits are configured to open upon the ostomy pack being filled, and an elastic material portion located on a back side of the garment and at least covering the one or more slits, wherein the elastic material portion is located adjacent to the front of the ostomy pack.

A third embodiment, illustrates a garment that includes an ostomy support garment in the form of a material having an OEP that includes one or more slits configured to be adjacent to an ostomy pack of a user, wherein the one or more slits are configured to open upon the ostomy pack being filled, and an elastic material portion located on a back side of the garment and at least covering a majority of slits, wherein the elastic material portion is located adjacent to the front of the ostomy pack.

### BRIEF DESCRIPTION OF THE DRAWINGS

FIG. 1 is an illustrative example of an outside view of a garment that is stretched.

FIG. 2 is an illustrative example of an outside view of garment that is unstretched.

FIG. 3 is an illustrative example of the stretch panel from inside the garment.

### DETAILED DESCRIPTION

Embodiments of the present disclosure are described herein. It is to be understood, however, that the disclosed embodiments are merely examples and other embodiments can take various and alternative forms. The figures are not necessarily to scale; some features could be exaggerated or minimized to show details of particular components. Therefore, specific structural and functional details disclosed herein are not to be interpreted as limiting, but merely as a representative basis for teaching one skilled in the art to variously employ the embodiments. As those of ordinary skill in the art will understand, various features illustrated and described with reference to any one of the figures can be combined with features illustrated in one or more other figures to produce embodiments that are not explicitly illustrated or described. The combinations of features illustrated provide representative embodiments for typical applications. Various combinations and modifications of the features consistent with the teachings of this disclosure, however, could be desired for particular applications or implementations.

A “ostomy expansion panel” and/or “ostomy extension portion” (e.g. “OEP”), was developed for persons with an ostomy. The OEP may help an entire segment of the population (more than 500,000+ in the U.S. alone) whose clothing needs may have been under appreciated. It allows for everyday clothing, such as slacks and skirts, to be tailored to solve the ostomates clothing needs by giving most fabrics the ability to ‘stretch’ as needed to accommodate the ostomy pouch or ostomy pack.

The advantage of the OEP is that it will allow for an ostomate to have more variety and clothing choices. An ostomate may attend regular daily activities, such as go to church, to business meetings, are professionals, etc., and would appreciate having not limited clothing options and to be able to dress in something other than jogging pants and spandex. The OEP may provide the ostomate confidence in their appearance by providing them suitable clothing for every occasion. For them to be fashionable and comfortable and at an affordable price.

An OEP may be developed into a garment such as a pants, dress, shorts, shirt, or any type of clothing. The OEP may or may not be developed in unison with the same material as the garment itself. For example, a denim jean may not be developed with a denim OEP. In another example, the material may be different. The OEP may be developed in unison with the garment during manufacturing, or inserted as a patch after the garment has been made. The garment may also include an area that utilizes a stretch material, such as cotton shirting or stretch cotton poplin (e.g., cotton and spandex/lycra blend), jersey knits, neoprene rubber, Lycra, Spandex, Elastane, nylon, chiffon, cotton jacquard, cotton sateen, stretch denim (e.g., includes spandex and cotton), stretch lace, stretch satin, stretch silk lining, stretch tulle, stretch viscose jacquard, stretch wool, stretch velvet, woolen jersey fabric, etc.

The OEP may be any shape or size in one embodiment. For example, the OEP may be Prolate Spheroid shaped like a football, round, oval, square, rectangular, or any other shape. This may coincide with the design or shape of the ostomy pack.

The OEP may be an area pre-selected on the garment during manufacturing and will usually be located on one side of the garment, but can be on both sides or centered. For example, the OEP may be located on the left side or right side adjacent to pockets, in one embodiment. In garments without pockets, the OEP may be located on the left side or right side adjacent to the abdomen area or high waist area. In another embodiment, the OEP may be on both the left and right side. In another embodiment, the OEP may be located in the center of the garment.

It will also be available as an unattached patch that can be inserted into an existing garment. Keeping in mind the shape of the OEP from the outside of the garment may appear as a series of vertical lines when the OEP is unstretched.

The length, spacing, and number of vertical lines will vary for each garment. Such variables may be dependent upon the size of the garment. In one embodiment, the center line may be the longest and the lines will decrease in length uniformly to each side in about 1 inch increments, however any increment in sizing may be used (e.g. ¼ inch, ½ inch, ¾ inch, etc.). The shortest lines on each may be half the center line length in one embodiment, however, the shortest lines may be smaller in any increment. The vertical lines may be spaced an inch apart in one embodiment, however, any sized spacing may be used (e.g. ¼ inch, ½ inch, ¾ inch, etc.).

When the OEP is stretched each vertical line may stretch open, allowing for the expansion of the ostomy pouch. The maximum amount of stretch across the entire panel will vary, dependent upon the maximum additional inches of expansion is desired. For example, in one embodiment the stretch across the entire panel may be no more than four inches. The inside of the garment is where the stretch fabric patch may be attached that enables the vertical openings to stretch open and recover to close back up. One embodiment may include the vertical openings with the stretch fabric attached directly to the perimeter of the entire opening giving the appearance of a very low profile.

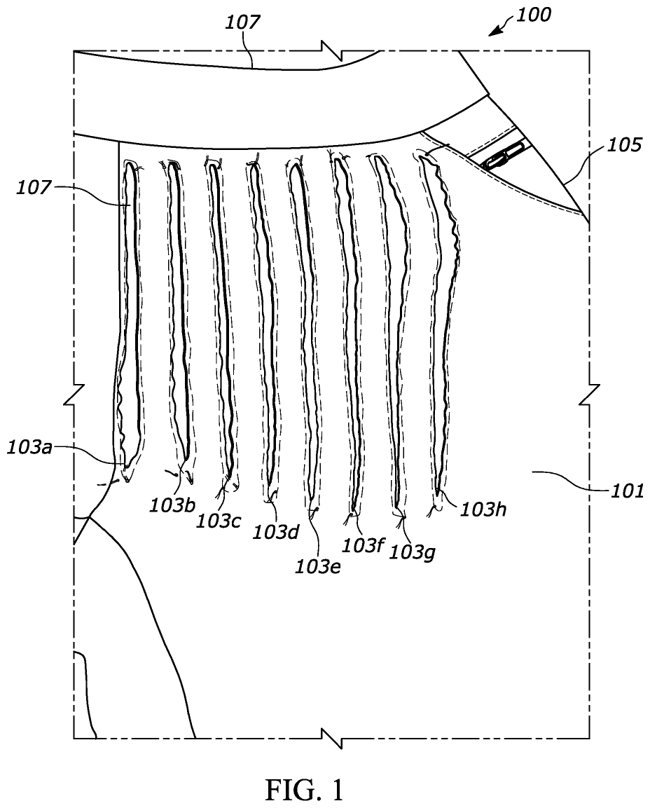

FIG. 1 is an illustrative example of an outside view of a garment 100 that is stretched. The garment may refer to any type of garment, such as a pair of pants, a dress, skirt, overalls, yoga pants, jumpsuits, culottes, tights, leggings, trousers, dungarees, dress pants, etc. The garment 100 may be any material and may include a top waist portion 107. The garment 100 may include one or more slits 103a, b, 103c, 103d, 103e, 103f, 103g. The slits 103 may provide breathability for an ostomy pack when it is filled or contracted. The slits 103 may run top to bottom in one embodiment and be parallel to one another. In another embodiment, the slits 103 may intersect or touch each other. In one embodiment, the OEP should give a garment 100 the ability to stretch at least 8″. However, other embodiments may allow for more or less stretch, such as 6″, 7″, 9″, 10″, etc. Each slit should have a maximum amount of stretch-ability, I′ll guess ½″. Therefore there would be 16 slits to allow for 8″ of stretch. Thus, the slits 103 will stretch as the ostomy pack is filled. The slits 103 may expand to reveal an elastic material 107. The elastic material may have a front side exposed to the slit opening 103 and a back side that will abut or touch an ostomy pack of a user. The elastic material 107 may be used to help with expansion and contraction of the ostomy pack. Furthermore, the number of slits may vary in each garment and in size. Thus, the number of slits may be different in numbers and size (e.g., thickness). Thus, the thickness of slit may impact the number of slits in the garment. For certain denser materials, such as denim, the slits 103, 201 may allow for expansion of the ostomy pack even in the case of form fitting clothing.

The slits 103 may be located on a front portion 101 of a garment. While the slits 103 may be located in various areas, they are generally going to be located in front of the ostomy of an ostomate. Such a location may be near the abdomen of a user on a left or right side, however, in other embodiments it may be centered near the person. Thus, the locations of the slits 103 may include adjacent a pocket 105 of the garment but below a waist portion 107. It should be noted that the slits may be found in any combination of areas that work with respect to the garment, the user, and the ostomy pack.

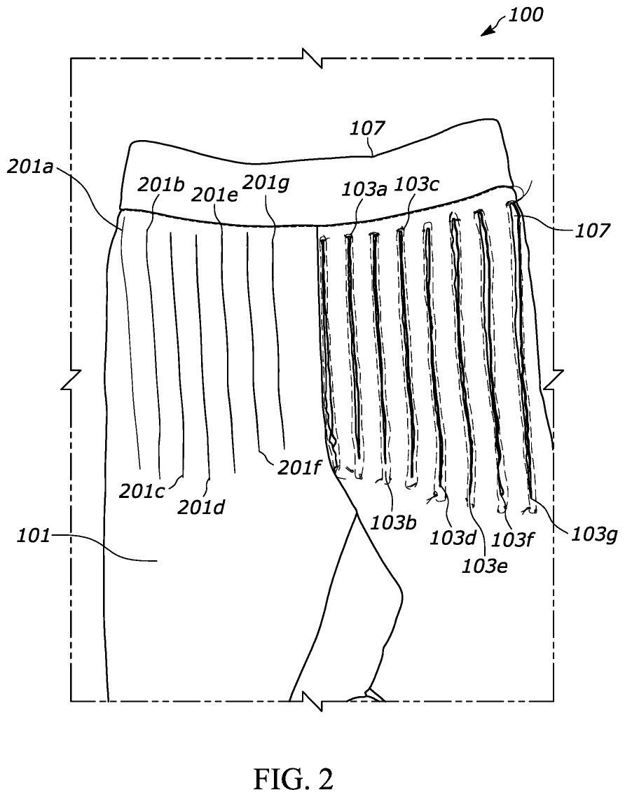

FIG. 2 is an illustrative example of an overview of garment that is unstretched. The garment 100, may be shown that has slits 201a, 201b, 201c, 201d, 201f, 201g located on a right abdominal portion of a person. The garment 100 may include one or more slits 103a, b, 103c, 103d, 103e, 103f, 103g located on the abdominal portion of a person. Thus, the slits 201 may be shown to view that the garment 100 may not have visible openings of the slits 201 in normal wear or use. Additionally, many users may wear clothing to cover or conceal the slits 103 and 201. In one embodiment, the various slits 201, and 103 may be spaced evenly and distributed evenly. However, in another embodiment the slits 201 and 103 may be spaced irregularly. The garment 100 in some embodiments may include an elastic waist band located below or at the waist portion 107. In an embodiment, the slits may be manufactured in a way to limit the view of the elastic material if the ostomy pack is not filled or filled minimally.

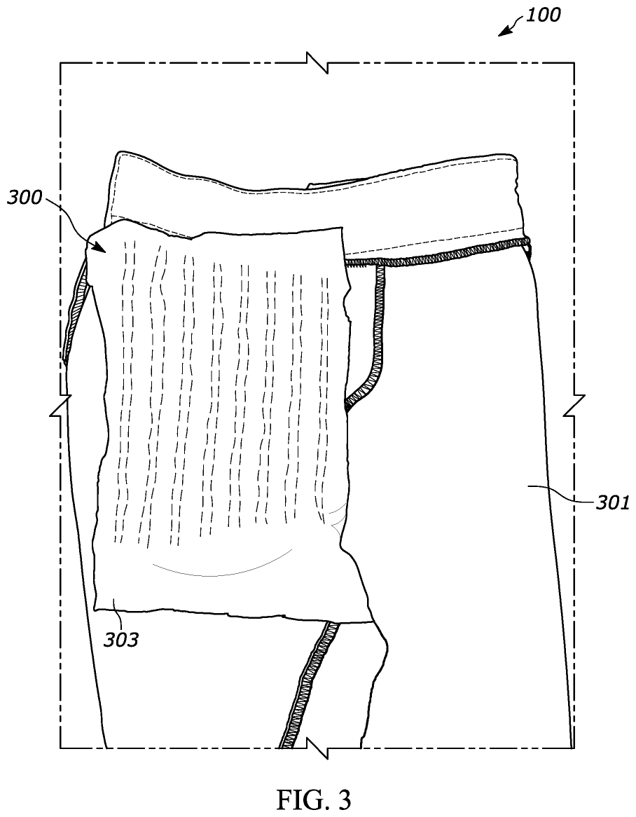

FIG. 3 is an illustrative example of the stretch panel from inside of the garment. The stitching should be close to the edge of the slit. The stitching of the elastic portion 303 may be located around the edge of the elastic material 303. The elastic material 303 may be attached to any method besides stitching. This may include a fastening element that attaches to the back of the garment via sewing, utilizing permanent fabric glue, permanent double sided fabric tape, a zip fastener, snaps, buckles, buttons, rings or mating elements, etc. The elastic portion 303 should mitigate any bunching up. The garment may also include an area that utilizes a stretch material. Thus the elastic portion may include such as cotton shirting or stretch cotton poplin (e.g., cotton and spandex/lycra blend), jersey knits, neoprene rubber, Lycra, Spandex, Elastane, nylon, chiffon, cotton jacquard, cotton sateen, stretch denim (e.g., includes spandex and cotton), stretch lace, stretch satin, stretch silk lining, stretch tulle, stretch viscose jacquard, wool, or stretch wool, stretch velvet, woolen jersey fabric, etc.

While exemplary embodiments are described above, it is not intended that these embodiments describe all possible forms encompassed by the claims. The words used in the specification are words of description rather than limitation, and it is understood that various changes can be made without departing from the spirit and scope of the disclosure. As previously described, the features of various embodiments can be combined to form further embodiments of the invention that may not be explicitly described or illustrated. While various embodiments could have been described as providing advantages or being preferred over other embodiments or prior art implementations with respect to one or more desired characteristics, those of ordinary skill in the art recognize that one or more features or characteristics can be compromised to achieve desired overall system attributes, which depend on the specific application and implementation. These attributes can include, but are not limited to cost, strength, durability, life cycle cost, marketability, appearance, packaging, size, serviceability, weight, manufacturability, ease of assembly, etc. As such, to the extent any embodiments are described as less desirable than other embodiments or prior art implementations with respect to one or more characteristics, these embodiments are not outside the scope of the disclosure and can be desirable for particular applications.

What is claimed is:

1. A garment, comprising:
an ostomy support garment including a material having an ostomy extension portion (“OEP”) that includes one or more slits configured to be adjacent to an ostomy pack of a user, wherein the one or more slits are configured to open upon the ostomy pack being filled; and
an elastic material portion located on a back side of the garment and at least covering the one or more slits, wherein the elastic material portion is located adjacent to a front of the ostomy pack.

2. The garment of claim 1, wherein the OEP is made from an elastic material different than a garment material, wherein the elastic material is configured to allow an increase and decrease of a perimeter of the one or more slits.

3. The garment of claim 1, wherein the garment is a first material and the elastic material is a different material.

4. The garment of claim 1, wherein the elastic material portion covers a majority of the one or more slits.

5. The garment of claim 1, wherein the elastic material portion is an elastic band, cord fabric, or cavalry twill.

6. The garment of claim 1, wherein the elastic material portion is connected via a fastening element to the garment enabling a closure around the perimeter of at least a top of the one or more slits.

7. The garment of claim 1, wherein a fastening element attaches at an edge of the one or more slits.

8. The garment of claim 1, wherein the elastic material portion attaches to the back of the garment via sewing, permanent fabric glue, permanent double sided fabric tape, a zip fastener, snaps, buckles, buttons, rings or mating elements.

9. A garment, comprising:
an ostomy support garment that includes one or more slits configured to be adjacent to the front of an ostomy pack of a user, wherein the one or more slits are configured to open upon the ostomy pack being filled; and
an elastic material portion located on a back side of the garment and at least covering the one or more slits, wherein the elastic material portion is located adjacent to the front of the ostomy pack.

10. The garment of claim 9, wherein the elastic material portions covered all of the one or more slits.

11. The garment of claim 9, wherein the one or more slits includes each and every slit being an identical dimension.

12. The garment of claim 9, wherein the one or more slits includes each and every slit being a different dimension.

13. The garment of claim 9, wherein the one or more slits includes each and every slit being a different length.

14. The garment of claim 9, wherein the one or more slits includes each and every slit being a different width.

15. A garment, comprising:
an ostomy support garment that includes one or more slits are adjacent an abdominal region of a user when the garment is worn, wherein the one or more slits are configured to open upon a ostomy pack being filled; and
an elastic material portion located on a back side of the garment and at least covering a majority of the one or more slits.

16. The garment of claim 15, wherein the elastic material portions covers all of the one or more slits.

17. The garment of claim 15, wherein the one or more slits are configured to open incrementally upon the ostomy pack being filled.
